(19) United States
(12) Patent Application Publication
(10) Pub. No.: US 2005/0186230 A1
(43) Pub. Date: Aug. 25, 2005

(54) Elemene compositions containing liquid oil

(75) Inventor: Andrew Xian Chen, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: SD Pharmaceuticals, Inc., Carlsbad, CA (US)

(21) Appl. No.: 11/040,079

(22) Filed: Jan. 21, 2005

Related U.S. Application Data
(60) Provisional application No. 60/538,851, filed on Jan. 23, 2004.

(51) Int. Cl. A61K 31/355; A61K 31/01; A61K 9/00

(52) U.S. Cl. 424/400; 514/762; 514/458

(57) ABSTRACT

The present invention provides injectable water-in-oil emulsions that comprise elemenes (preferably, bete-elemene), liquid oil, an emulsifier (optionally) and a pharmaceutically acceptable aqueous phase. The present invention also provides methods for making and using such emulsions.

### CROSS-REFERENCE TO RELATED APPLICATION

This application claims the benefit of U.S. Provisional Patent Application No. 60/538,851 filed Jan. 23, 2004; which is incorporated herein by reference in its entirety.

### BACKGROUND OF THE INVENTION

1. Field of the Invention

The present invention relates generally to pharmaceutical compositions that comprise elemene(s).

2. Description of the Related Art

Elemene is a chemical compound that can be extracted from numerous plants. Three isomers are found, namely, alpha-, beta- and gamma-elemene, with the beta-elemene being the most important form therapeutically. Beta-elemene has the chemical name of 1-methyl-1-vinyl-2,4-diisoprotenyl-cyclohexane. The structure, formula and molecular weight of beta-elemene are as below:

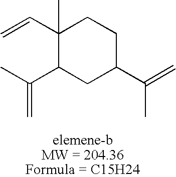

Therapeutically, mixtures of all three elemenes have been used with beta-elemene being the predominant form.

More than 50 different plants have been found to contain elemene, such as Radix Inulae, Radix Ginseng, E. Wenyujin chen et C. Ling and others. They grow in tropical areas around the world. Elemenes are commonly extracted from Curcuma Wenyujin chen et C. Ling, Curcuma aromatia, and Curcuma longa Inn (all belonging to Ziniberaceae).

The extraction methods of elemenes have been disclosed in U.S. Patent Application No. 2003216605, U.S. Pat. No. 6,464,839, Chinese Patent Application Publication Nos. 1413580, 1408348, 1408347, and 1200266.

Preparations made from Curcuma Aromatica Salisb (which contains elemene) have been a part of Chinese herbal remedies for centuries. It has been used internally and topically for a wide variety of ailments.

Elemenes at various concentrations with other ingredients appear in applications as diverse as a mosquito repellent (see U.S. Pat. No. 5,66,781), burn treatment (see U.S. Pat. Nos. 5,558,914 and 5,384,125) and treatment for Herpes Simplex (see U.S. Pat. No. 5,385,733).

Of particular interest is the anti-tumor characteristics exhibited by elemenes. The majority of reports of the anti-tumor activity of elemenes is found in Chinese scientific journals. In December 1993, beta-elemene was designated as a Chinese national Class II new drug. In February 1994, the anti-cancer effect of beta-elemene was confirmed by the health authority of China. After that, an injectable and an oral product of elemenes have been approved for marketing as anticancer drugs by the Chinese Drug Authority (State Food and Drug Administration, SFDA) and are available commercially.

The marketed injectable elemene product is manufactured by Dalian JinGang Pharmaceutical Co., Ltd. and is indicated for the first line treatment of malignant pleural and peritoneal effusions (chest and abdominal ascites), malignant brain tumors, cancers of respiratory and digestive tracks, and for the second line treatment of cancers of female reproductive organs, breast cancer, metastatic bone cancer, skin cancer, lymphoma, and leukemia according to the product package insert (Elemene Emulsion Product Brochure by Dalian JinGang Pharmaceutical Co., Ltd.).

During the 2 year clinical trial of the drug in China, it was determined that elemene has not only the ability to manage malignant chest and abdominal ascites, but also had beneficial effects on brain tumors (neuroglioma), liver and esophageal cancers.

Wang et al. (Clinical trial-Journal article—NIH data base ID#98048551—“Phase III clinical trial of elemenum emulsion in the management of malignant pleural and peritoneal effusions”, Zhonghua Zhong Liu Za Zhi, 1996 November; 18(6): 464-7) reported the use of elemene emulsion in the management of malignant effusions in a multicenter phase III clinical trial with 484 patients, including 313 with pleural effusion and 171 with peritoneal effusion. The response rates were 77.6% in patients with malignant pleural effusion, and 66.1% in patients with peritoneal effusion. There were no noticeable bone marrow, liver, cardiac and renal toxicities; while fever, local pain and gastrointestinal reaction were the major adverse effects. It should be noted that 27% of the cerebral carcinoma patients reached a complete remission level with a combination of the elemene and local chemotherapy in the expanded clinical trials.

Tan et al. (Tan, P, Zhang W, Cai W. “Clinical study on treatment of 40 cases of malignant brain tumor by elemene emulsion injection”, Zhongguo Zhong Xi Yi He Za Zhi. 2000 Sep. 20 (9): 645-8) reported clinical study on treatment of 40 cases of malignant brain glioma by elemene emulsion injection. The results indicated that elemene had significant effect on treatment of malignant brain tumor with 4 cases of complete remission, 26 cases of partial remission and a total effective rate of 75%.

Work performed in the Department of Embryology of the Dalian Medical University revealed that beta-elemene is able to pass through the blood brain barrier (BBB) and thus reach tumors within the brain.

Shi, J. (Journal article—NHI data base ID # 82631581—“Experimental pharmacological studies on the volatile oil of Wen-E-Zhu (Curcuma Aromatica Salisb): Study on the anti-tumor activity of beta-elemene”—Zhongyao Tongbao, Luda Inst. Medical and Pharmaceutical Sciences, Luda, P.R.China) demonstrated that the beta-elemene component of elemene exhibited marked anti-tumor activity against murine Ehrlich ascites carcinoma and rat ascitic reticulum cell sarcoma. Diarrhea and weight loss were reported as side effects during treatment. The material used contained beta-elemene 65%, gamma and delta-elemene 20%, and unknowns 15%.

Yang et al. (H., Wang, X. and Yu, L, Journal article—NHI database ID# 98048492—“The antitumor activity of elemene is associated with apoptosis”, Chung Hua Chung Liu Tsa Chih; 18(3): 169-72 1996, Cancer Institute, Zhejiang Medical university, Hangzhou, China) determined that the anti-tumor activity of elemenes is associated with cell cycle arrest from S to G₂M phase transition and with the induction of apoptosis. They further demonstrated this effect in vitro and in vivo to human and murine tumor cells. The in vitro IC₅₀ of elemene was 27.5, 81 and 254.3 μg/mL, for promyelocytic leukemia HL 60 cells, erythroleukemia K562 cells, and peripheral blood leukocytes (PBL), respectively.

Zou et al. (Zou L, Liu W, Yu L. “Beta-elemene induces apoptosis of K562 leukemia cells” Zhonghua Zhong Liu Za Zhi. 2001 May; 23(3): 196-8) reported beta-elemene induces apoptosis of K562 leukemia cells and concluded that beta-elemene exerts its cytotoxic effect on K562 leukemic cells by the induction of apoptosis.

Yuan et al. (Yuan J, Gu Z L, Chou W H, Kwok C Y, “Elemene induces apoptosis and regulates expression of bcl-2 protein in human leukemia K562 cells”, Zhongguo Yao Li Xue Bao. 1999 February; 20(2): 103-6) also demonstrated that elemene induces apoptosis of K562 cells, which is related with the down-regulation of bcl-2 protein in K562 cells.

Guo (Journal article—NIH data base ID# 83285582—“lsolation and identification of elemene from the essential oil of Curcuma Wenjujin”, Chung Yao Tung Pao; 8(3): 31 1983) described the isolation of elemene from Curcuma Wenyujin and described elemene's anti-neoplastic activities. Beta-elemene in a concentration of 92% of the total elemenes was used.

Fu (Journal article—NIH data base ID #84282970—“Antitumor effect and pharmacological actions of beta-elemene isolated from the rhizome of Curcuma aromatica”, Chung Yao Pao; 9(2): 35-9 1984) reported on the anti-tumor effect of beta-elemene.

An elemene emulsion for oral administration has also been recently approved for gastric cancer and other cancers of digestive tract (Oral Elemene Emulsion Product Brochure by Dalian JinGang Pharmaceutical Co., Ltd.).

Since elemenes are lipophilic and insoluble in water, they must be solubilize or dispersed appropriately in an aqueous vehicle in order for intravenous administration.

Chinese Patent Application Publication No. 1221607 is related to liposoluble medicinal liposome production technique and elemene liposome injection. The raw materials used including phosphatide, cholesterin and liposoluble medicine. The formed elemene lipoplast injection contains phosphatide (1-4 wt. %), cholesterin (0.3-2 wt. %) and elemene (0.5-1.5 wt. %). No oil was used. This type of composition was defined as the liposome. The degree of drug incorporation in this formulation was not revealed.

Chinese Patent Application Publication No. 10766-13 is directed to elemene emulsion injection and its preparing process. The elemene emulsion injection is prepared with elemene, refined soybean lecithin, cholesterin and phosphate buffering liquid. It does not contain any triglycerides or other liquid oil components. The degree of drug incorporation in this formulation was not revealed.

Chinese Patent Application Publication No. 1244389, entitled “Elemi olefine injecta and its preparation,” relates to an elemene injection. According to this patent, Elemene and its isomer is mixed with propanediol, Cremophor EL through stirring, and the mixture is further heated, filtered, packaged, inflated with nitrogen, melting sealed and disinfected to produce the injection. Cremophor EL can cause severe hypersensitivity reaction if given intravenously and is thus undesirable.

Chinese Patent Application No. 1508176 relates to an elemene formulation based on the use of hydroxypropyl-bete-cyclodextrin. The patent states that elemene and hydroxypropyl-bete-cyclodextrin form an inclusion complex allowing elemene to be water soluble. The final dosage forms base on this inclusion complex include suppository, topical cream, tablet and capsule. The formulation is not related to this application which does not use cyclodextrin or inclusion complex.

The marketed Elemene for Injection drug product (“the marketed formulation”) is an “emulsion” containing soy phospholipid, cholesterol and elemenes. Because combinations of soy phospholipid and cholesterol are customarily used to fabricate bilayer membrane structures in liposome preparations, and because the marketed formulation does not contain any liquid oil component, such a formulation is a liposome formulation, not an oil-in-water emulsion. The particles that give the marketed formulation an appearance of an emulsion are solid particles (not oil droplets like in an oil-in-water emulsion), and the marketed formulation is more likely a solid dispersion or suspension system than an oil-in-water emulsion.

Moreover, the marketed formulation is known to cause severe vein irritation or phlebitis if administered into peripheral veins according to the Elemene Emulsion for Injection Product Brochure by Dalian JinGang Pharmaceutical Co., Ltd. Therefore, this product is given by intraperitoneal injection (IP) or through a carotid artery catheter into the carotid artery, both methods are risky, painful and inconvenient and require special skill and some surgical intervention. It has also been reported that the marketed formulation caused severe abdominal pain, accompanied with a drop in blood pressure, agitation and perspiration in cancer patients, following the intraperitoneal injections.

In light of these problems confronting the use of elemenes for medical treatments, it can be seen that a need exists in the art for a pharmaceutical composition that comprises elemene(s) and is generally stable, safe and non-irritating.

### BRIEF SUMMARY OF THE INVENTION

This invention generally relates to pharmaceutical compositions of an injectable elemene oil-in-water emulsion and methods of producing and using the emulsion. The compositions of the present invention, which comprise at least one liquid oil, are much less vein irritable and more stable than elemene compositions that do not contain any liquid oil.

In one aspect, the present invention provides an injectable oil-in-water emulsion that comprises a therapeutically effective concentration of elemene(s) (and/or pharmaceutically acceptable salt(s), complex(es) and ester(s) thereof) and at least one liquid oil. Elemenes may be alpha-, beta-, gamma-elemene, or a mixture thereof. In certain embodiments, the present composition contains above 50%, 60%, 70%, 80% or 90% of beta-elemene based on the weight of all elemene isomers present.

In certain embodiments, the emulsion contains elemene(s) (and/or pharmaceutically acceptable salt(s), complex(es) and ester(s) thereof) at a concentration between about 0.5 mg/mL to about 50 mg/mL (including any value within this range). In certain embodiments, the emulsion contains elemene(s) (or pharmaceutically acceptable salt(s), complex(es) and ester(s) thereof) at a concentration of about 0.1% to about 5% by weight (including any value within this range).

In certain embodiments, the liquid oil comprises a monoglyceride, a diglyceride, a triglyceride, or a mixture thereof. In certain other embodiments, the liquid oil comprises a vitamin E or a derivative thereof.

In certain embodiments, the liquid oil comprises vegetable oil. In certain embodiments, the liquid oil comprises a mixture of long chain triglyceride(s) (e.g., vegetable oil) and medium chain triglyceride(s). In certain embodiments, the weight ratio of the long chain triglyceride(s) to the medium chain triglyceride(s) is about 9:1 to about 1:9.

In certain embodiments, the present composition comprises a liquid oil at a concentration between about 2% to about 40% (including any value with this range).

The present composition may further comprise one or more of the following components: an emulsifier (e.g., an egg or soy lecithin), a tonicity-adjusting agent (e.g., glycerol, mannitol, sucrose, glucose, trehloase, glycine, and propylene glycol), an antimicrobial preservative (e.g., disodium edetate), and a stabilizing agent (e.g., disodium edetate).

In certain embodiments, the present composition comprises an emulsifier at a concentration between about 1% to about 5% (including any value within this range).

In certain embodiments, the present composition comprises a tonicity-adjusting agent at a concentration between about 1% to about 10% (including any value within this range).

In certain embodiments, the present composition comprises disodium edetate. In certain specific embodiments, disodium edetate is at a concentration of about 0.001% to about 0.1%.

In certain embodiments, the pH of the aqueous phase in the present oil-in-water emulsion is about 5 to about 8 (including any value within this range).

In certain embodiments, the average size of the oil droplets in the emulsion is less than about 500 nm, 400 nm, 300 nm, 200 nm, 150 nm, or 100 nm.

In certain embodiments, the concentration of elemene (or a pharmaceutically acceptable salt, complexes or ester thereof) in the aqueous phase of the present oil-in-water emulsion is no more than about 2 μg/mL to about 30 μg/mL (including any value within this range).

The compositions provided by the present invention are generally stable. In certain embodiments, the present composition may be stored under appropriate conditions (e.g., at 2-8° C. or 25° C.) for an extended period of time (e.g., for at least one month, two months, three months, four months, five months, six months or longer) without change (e.g., increase or decrease) in the average size of the oil droplets in the emulsion by more than 50% of the original average size. In certain embodiments, the present composition may be stored under appropriate conditions (e.g., at 2-8° C. or 25° C.) for an extended period of time (e.g., for at least one month, two months, three months, four months, five months, six months or longer) without change (e.g., increase or decrease) in the elemene concentration in the aqueous phase by more than 50% of the original concentration.

The compositions provided by the present invention are generally non-irritating and safe when administered intravenously. Injections of the present compositions do not cause any significant pain, irritation, inflammation or phlebitis at the tissue near the injection site. In addition, injections of the present compositions do not cause any significant systemic toxicity or hypersensitivity reactions to any of the components of the compositions.

In another aspect, the present invention provides a lyophilized formulation of elemene(s), wherein the formulation, when hydrated, produces an oil-in-water emulsion as described herein.

In certain embodiments, the average droplet size of the rehydrated emulsion is no more than about 300%, 200% or 150% of the average droplet size of the emulsion before the freeze-drying.

In another aspect, the present invention provides methods for using the above compositions. Because the present compositions are effective means for delivering elemenes (and/or pharmaceutically acceptable salts, complexes and esters thereof), they may be used for treating any diseases or disorders that are responsive to elemene treatments. For instance, the present compositions may be used to treat various malignancies, especially malignant pleural and peritoneal effusions (chest and abdominal ascites), malignant brain tumors, cancers of respiratory and digestive tracks (e.g., esophageal cancer and gastric cancer), cancers of female reproductive organs, breast cancer, metastatic bone cancer, skin cancer, lymphoma, leukemia, and liver cancer.

Throughout this application, various publications are referenced and full citations for these publications may be found in the text where they are referenced. The disclosures of these publications are hereby incorporated by reference into this application in their entities.

### DETAILED DESCRIPTION OF THE INVENTION

The present invention, in one aspect, provides pharmaceutical compositions for delivering elemene(s) and/or pharmaceutically acceptable salts, complexes and esters thereof. Such compositions are oil-in-water emulsions that comprise elemene(s) (and/or pharmaceutically acceptable salts, complexes and esters thereof), at least one liquid oil, and water. Optionally, these compositions of the present invention may each further comprise an emulsifier, a tonicity modifier, and/or stabilizer. The compositions of the present invention have one or more of the following properties: (1) injectable, (2) stable under appropriate storage conditions, (3) vein non-irritable, (4) containing elemene(s) (and/or pharmaceutically acceptable salts, complexes and esters thereof) at pharmaceutically effective concentration, (5) sterilizable by filtration, (6) containing components acceptable by regulatory agencies (e.g., the U.S. FDA), and (7) not causing hyperlipodemia or other side effects.

Elemenes are small and lipophilic molecules belonging to the terpene family. Physically, they behave like volatile oil with a high vapor pressure at room temperature. This inventor found that elemene oil is a strong solvent capable of dissolving or swelling many plastic or rubber components. Elemene oil can also extract plasticizers out of a rubber or plastic closures. Being small molecules with a high vapor pressure, elemene oil is very “invasive” and capable of penetrating into plastic or rubber material, and possibly, tissues as well. The strong tendency of causing severe irritation to the tissues near the injection site of elemenes may be attributable to their good solvent property and the “invasive” nature.

For vein irritating drugs, a variety of approaches have been taken to address this problem, including attempts to shield the irritating drugs in a carrier system. The underlining thinking is that by incorporating the drug into such a carrier system, it is possible to minimize the direct contact of the vein endothelium tissue with the irritating free drug molecules, thus reducing the exposure of the local tissues at the injection site to the drug.

However, the previously known elemene formulation (e.g., the marketed “emulsion” formulation that contains elemenes, soy phospholipid, cholesterol and water) failed to provide vascular protective effect against elemene irritation problems. This inventor found that despite of their lipophilicity, elemenes still have some solubility in water, and that the marketed formulation contains elemenes in the aqueous phase in an amount sufficient to cause irritation. In other words, the marketed formulation of elemene failed to incorporate elemenes completely into the carrier particles; therefore the irritating drug is not properly “shielded.”

To correct the vein irritation problem, this invention discloses improved formulation compositions for elemenes for injections. The new elemene formulation compositions have demonstrated a greatly improved venous vascular tolerability in the tested animals. More specifically, the present invention provides injectable oil-in-water emulsions having a therapeutically effective concentration of elemenes and including at least one liquid oil. Without wishing to be bound, the present inventor believes that the inclusion of the liquid oil allows more elemene to be incorporated into the oil phase, and thus reduces the concentration of elemene in the aqueous phase and irritation to a host.

An “oil-in-water emulsion” refers to a system with a liquid oil phase and an aqueous phase where the liquid oil phase (the discrete phase), in the form of small liquid oil droplets, is dispersed in the aqueous phase (the continuous phase).

In certain embodiments, the present compositions comprise elemene isomers, such as beta elemene, which is effective in treating cancer. “Beta elemene” refers to 1-methyl-1-vinyl-2,4-diisoprotenyl-cyclohexane (see U.S. Pat. No. 6,464,839) of natural or synthetic origins known to the art. In certain embodiments, the present composition contains at least 50%, 60%, 70%, 80% or 90% of beta-elemene based on the weight of all elemene isomers present.

The present application also provides injectable oil-in-water emulsions of pharmaceutically acceptable salts, complexes and esters of elemene(s). By “pharmaceutically acceptable salts, complexes and esters” as used herein is meant those salts, complexes and esters which are, within the scope of sound medical judgment, suitable for use in contact with the tissues of humans and lower animals without undue toxicity, irritation, allergic response, and the like, commensurate with a reasonable benefit/risk ratio, and effective for their intended use (such as in the anticancer treatment and prophylaxis). Exemplary pharmaceutically acceptable salts, complexes and esters of elemenes include, but not limited to sodium salt, potassium salt, calcium salt, ammonium salt, hydrochloride salt, phosphate salt, acetate salt, lactate salt, succinate salt, citrate salt, sulfate salt, methasulfonate salt, acetate ester, succinate ester, lactate ester, cyclodextrin complex, polyvinyl pyrrolidine complex, polyethylene glycol complex, nicotinamide complex, etc.

Because the compositions of the invention offer improved tissue compatibility for elemene(s) and pharmaceutically acceptable salts, complexes and esters, these compounds can be administered at a therapeutically effective concentration into a peripheral vein with greatly reduced irritation at the injection site.

By “tissue compatibility” as used herein is meant that pain, irritation, inflammation or other adverse reactions at the injection site to the intravenous injection of the elemene compositions is well tolerated by the patient so that no special care for the injection site is needed.

By “therapeutically effective concentration” as used herein is meant the concentration of elemene(s) (or pharmaceutically acceptable salts, complexes and esters thereof) that is effective to treat or prevent diseases or disorders such as elemene susceptible cancers, at a reasonable benefit/risk ratio applicable to any medical treatment. An exemplary therapeutically effective concentration of elemenes) is from about 0.5 mg/mL to about 50 mg/mL (including any value with this range, such as 5 mg/mL).

In certain embodiments, the emulsion contains elemene(s) (or pharmaceutically acceptable salts, complexes and esters thereof) at a concentration of about 0.1% to about 5% by weight (including any value within this range).

“Concentration by weight” (wt. % or w/w), as used herein, refers to the ratio (in percentage) of the weight of a component (e.g., an elemene) of a composition (e.g., an elemene oil-in-water emulsion) to the total weight of the composition, if not otherwise noted.

The total daily dose of the present compositions will be decided by the attending physician within the scope of sound medical judgment. The specific total daily dose level for any particular patient will depend on a variety of factors including age, body weight, general health, sex, diet, time of administration, the severity of the particular disease undergoing therapy, and like factors well known in the medical arts. Under certain conditions, the therapeutically effective daily dose of elemenes may be from about 400 mg to about 600 mg.

As described above, the present compositions comprise at least one liquid oil. By “liquid oil” as used herein is meant pharmaceutically acceptable, water-immiscible and injectable oleaginous vehicles that are liquid at room temperature (20-25° C.) or that have a melting point below 20° C. The “liquid oil” used herein comprises glyeride oil, vitamin E or tocopherol oil or the like.

By “glyceride oil” as used herein is meant a glycerol ester of fatty acid which is liquid at room temperature (20-25° C.), and which consists primarily of glycerol esters of C6 to C22 fatty acids. The glycerol ester can be mono-, di- or triglyceroldes and their mixtures, and the fatty acids can be of short chain (C6 to C8), medium chain (C8-C12), or long chain (C14 to C22) aliphatic fatty acids.

By “triglyceride oil” as used herein is meant a triglyceride composition which is liquid at room temperature (20-25° C.), and which consists primarily of triglycerides of C6 to C22 fatty acids. The triglyceride oil can be short chain (C6 to C8), medium chain (C8-C12), or long chain (C14 to C22) and preferably a mixture of the medium (C8-C12) and long chain C14-C18) aliphatic fatty acids.

In certain embodiments, the present compositions comprise vegetable oil. “Vegetable oil” refers to oil derived from plant seeds or nuts. Exemplary vegetable oils include, but are not limited to, almond oil, borage oil, black currant seed oil, corn oil, safflower oil, soybean oil, cottonseed oil, peanut oil, olive oil, rapeseed oil, coconut oil, palm oil, canola oil, sesame oil, etc.

Vegetable oils are typically “long-chain triglycerides,” formed when three fatty acids (usually about 14 to about 22 carbons in length, with unsaturated bonds in varying numbers and locations, depending on the source of the oil) form ester bonds with the three hydroxyl groups on glycerol. In certain embodiments, vegetable oils of highly purified grade (also called “super refined”) are generally used to ensure safety and stability of oil-in-water emulsions.

An example of vegetable oil is soybean oil, which typically has a fatty acid composition of about 80% oleic and linoleic acids. An injectable grade of soybean oil is commercially available as Super Refined USP grade oil from Croda Inc. of Parsippany, N.J. Another example of vegetable oil is safflower oil.

Also suitable for use in delivering elemene(s) are oil-in-water emulsions that are generally prepared from a mixture of neutral triglycerides containing predominantly unsaturated fatty acids. An example of such an emulsion is Liposyn II, a commercially available triglyceride oil and water emulsion. The major component fatty acids are linoleic, oleic, palmitic, stearic, and linolenic acids. In addition, these products can contain soy or egg yolk phospholipids as an emulsifier, and glycerol to adjust tonicity. The emulsified fat particles are generally about 200 to about 500 nanomers in diameter, similar to naturally occurring chylomicrons.

In certain embodiments, the present compositions comprise a mixture of medium chain triglyceride(s) (MCT) and long chain triglyceride(s) (LCT) (e.g., vegetable oil). Like vegetable oils, MCT's have been used extensively in emulsions designed for injection as a source of calories, for patients requiring parenteral nutrition. Such oils can be prepared synthetically by well-known techniques, or can be obtained from natural sources by known techniques of thermal or solvent fractionation of suitable natural oils, such as palm oil or coconut oil, to yield fractions rich in the desired low-melting triglycerides. An example of low-melting, low molecular weight triglyceride oil is a low molecular weight fraction of coconut or palm oil that is rich in mixed esters of caprylic (octanoic) and capric (decanoic) acids. Such oil is commercially available as Miglyol 812 from SASOL Germany GmbH, CRODAMOL GTCC-PN from Croda Inc. of Parsippany, N.J., or Neobees M-5 oil from PVO International, Inc., of Boonton, N.J. Other low-melting cuts of medium chain oil are also suitable.

Also suitable for use in delivering elemene(s) are oil-in-water emulsions that are generally prepared from a mixture of a vegetable oil (such as soybean or safflower oil) and a medium chain triglyceride. An example of such an emulsion is LIPOFUNDIN MCT, a commercially available medium and long chain triglyceride oil and water emulsion. In addition, these products can contain soy or egg yolk phospholipids as an emulsifier, and glycerol to adjust tonicity. The emulsified fat particles are generally about 200 to about 500 nanomers in diameter, similar to naturally occurring chylomicrons.

In the embodiments where the composition comprises a mixture of LCT(s) and MCT(s), the weight ratio of the LCT(s) to the MCT(s) may be about 9:1 to about 1:9 (including any ratio within this range).

In certain compositions according to this invention, the triglyceride oil may contain small amounts of mono- and/or diglycerides to enhance solubility of the components or to enhance emulsification. In other compositions of this invention, it will be preferable that the oil has a low polarity. In such a case, the triglyceride oils will be low in the content of mono- and diglycerides, as well as phospholipids, all of which have significant polarity.

In certain compositions according to this invention, the triglyceride oil may contain glycerol esters of C-20 or C-22 unsaturated fatty acids such as eicosapentaenoic acid (C-20:5) or docosahexanoic acid (C22:6), in the oil phase.

Other liquid oil useful in the present invention may be natural or synthetic tocopherol oil such as vitamin E acetate, vitamin E, and vitamin E succinate. Still other liquid oil useful in the present invention may be animal fat that is inherently liquid at room temperature, such as various fish oils. Still other liquid oil useful in the present invention may be a fatty acid that is inherently liquid at room temperature, such as oleic oil, linoleic acid, linolenic acid, palmitic acid, erucic acid, arachidonic acid, lauric acid, myristic acid, butyric acid, arachidic acid and stearic acid.

The liquid oil is generally present in a range of from about 2% to about 40% in the final emulsion formulation by weight (including any value within the range).

The present compositions may also contain an emulsifier. “Emulsifier” as used herein means a compound that prevents the separation of the injectable emulsion into individual oil and aqueous phases. Emulsifiers useful in the present invention generally are (1) compatible with the other ingredients of the oil-in-water emulsions of the present invention, (2) do not interfere with the stability or efficacy of the elemene(s) in the emulsions, (3) are stable and does not deteriorate in the preparation, and (4) are non-toxic.

Suitable emulsifiers include, but are not limited to, propylene glycol mono- and di-fatty acid esters, polyoxyethylene sorbitan fatty acid esters, polyoxyethylene fatty acid esters, polyoxyethylene-polyoxypropylene co-polymers and block co-polymers, salts of fatty alcohol sulphates, sorbitan fatty acid esters, esters of polyethylene-glycol glycerol ethers, oil and wax based emulsifiers, glycerol monostearate, glycerine sorbitan fatty acid esters and phospholipids.

A “phospholipid” refers to a triester of glycerol with two fatty acids and one phosphate ion. Exemplary phospholipids useful in the present invention include, but are not limited to, phosphatidyl chlorine, lecithin (a mixture of choline ester of phosphorylated diacylglyceride), phosphatidylethanolamine, phosphatidylglycerol, phosphatidic acid with about 4 to about 22 carbon atoms, and more generally from about 10 to about 18 carbon atoms and varying degrees of saturation. The phospholipid component of the drug delivery composition can be either a single phospholipid or a mixture of several phospholipids. The phospholipids should be acceptable for the chosen route of administration.

The phospholipids useful in the present invention can be of natural origin. Naturally occurring lecithin is a mixture of the diglycerides of stearic, palmitic, and oleic acids, linked to the choline ester of phosphoric acid, commonly called phosphatidylcholine, and can be obtained from a variety of sources such as eggs and soya beans. Soy lecithin and egg lecithin (including hydrogenated versions of these compounds) have a long history of safety, possess combined emulsification and solubilization properties, and tend to be broken down into innocuous substances more rapidly than most synthetic surfactants. Commercially available soya phospholipids are the Centrophase and Centrolex products marketed and sold by Central Soya, Phospholipon from Phospholipid GmbH, Germany, Lipoid by Lipoid GmbH, Germany, and EPIKURON by Degussa.

Phospholipids useful in the present invention can also be synthesized. Exemplary common synthetic phospholipids are listed below:

Diacylglycerols

1,2-Dilauroyl-sn-glycerol (DLG)
1,2-Dimyristoyl-sn-glycerol (DMG)
1,2-Dipalmitoyl-sn-glycerol (DPG)
1,2-Distearoyl-sn-glycerol (DSG)
   Phosphatidic Acids
1,2-Dimyristoyl-sn-glycero-3-phosphatidic acid, sodium salt (DMPA,Na)
1,2-Dipalmitoyl-sn-glycero-3-phosphatidic acid, sodium salt (DPPA,Na)
1,2-Distearoyl-sn-glycero-3-phosphatidic acid, sodium salt (DSPA,Na)
   Phosphocholines
1,2-Dilauroyl-sn-glycero-3-phosphocholine (DLPC)
1,2-Dimyristoyl-sn-glycero-3-phosphocholine (DMPC)
1,2-Dipalmitoyl-sn-glycero-3-phosphocholine (DPPC)
1,2-Dipalmitoyl-sn-glycero-3-phosphocholine (DPPC)
1,2-Distearoyl-sn-glycero-3-phosphocholine (DSPC)
1,2-Distearoyl-sn-glycero-3-phosphocholine (DSPC)
   Phosphoethanolamines
1,2-Dilauroyl-sn-glycero-3-phosphoethanolamine (DLPE)
1,2-Dimyristoyl-sn-glycero-3-phosphoethanolamine (DMPE)
1,2-Dipalmitoyl-sn-glycero-3-phosphoethanolamine (DPPE)
1,2-Distearoyl-sn-glycero-3-phosphoethanolamine (DSPE)
   Phosphoglycerols
1,2-Dilauroyl-sn-glycero-3-phosphoglycerol, sodium salt (DLPG)
1,2-Dimyristoyl-sn-glycero-3-phosphoglycerol, sodium salt (DMPG)
1,2-Dimyristoyl-sn-glycero-3-phospho-sn-1-glycerol, ammonium salt (DMP-sn-1-G,NH₄)
1,2-Dipalmitoyl-sn-glycero-3-phosphoglycerol, sodium salt (DPPG,Na)
1,2-Distearoyl-sn-glycero-3-phosphoglycerol, sodium salt (DSPG,Na)
1,2-Distearoyl-sn-glycero-3-phospho-sn-1-glycerol, sodium salt (DSP-sn-1 G,Na)
   Phosphoserines
1,2-Dipalmitoyl-sn-glycero-3-phospho-L-serine, sodium salt (DPPS,Na)
   Mixed Chain Phospholipids
1-Palmitoyl-2-oleoyl-sn-glycero-3-phosphocholine (POPC)
1-Palmitoyl-2-oleoyl-sn-glycero-3-phosphoglycerol, sodium salt (POPG,Na)
1-Palmitoyl-2-oleoyl-sn-glycero-3-phosphoglycerol, ammonium salt (POPG,NH₄)
   Lysophospholipids
1-Palmitoyl-2-lyso-sn-glycero-3-phosphocholine (P-lyso-PC)
1-Stearoyl-2-lyso-sn-glycero-3-phosphocholine (S-lyso-PC)
   Pegylated Phospholipids
N-(Carbonyl-methoxypolyethyleneglycol 2060)-MPEG-2000-DPPE
1,2-dipalmitoyl-sn-glycero-3-phosphoethanolamine, sodium salt
N-(Carbonyl-methoxypolyethyleneglycol 5000)-MPEG-5000-DSPE
1,2-d istearoyl-sn-glycero-3-phosphoethanolamine, sodium salt
N-(Carbonyl-methoxypolyethyleneglycol 5000)-MPEG-5000-DPPE
1,2-dipalmitoyl-sn-glycero-3-phosphoethanolamine, sodium salt
N-(Carbonyl-methoxypolyethyleneglycol 750)-MPEG-750-DSPE
1,2-distearoyl-sn-glycero-3-phosphoethanolamine, sodium salt
N-(Carbonyl-methoxypolyethyleneglycol 2000)-MPEG-2000-DSPE
1,2-distearoyl-sn-glycero-3-phosphoethanolamine, sodium salt
   Others
   Sodium Cholesteryl Sulfate SCS

The present invention also includes compositions containing other emulsifiers. Such emulsifiers include, but are not restricted to glycodeoxycholic or glycocholic acids or a combination thereof; and non-ionic surfactants. Exemplary non-ionic surfactants include polysorbate, sorbitan monostearate and combinations thereof and Poloxamer block copolymers. Poloxamer block copolymers are synthetic copolymers of ethylene oxide and propylene oxide represented by the following chemical structure:
HO(C₂H₄O)a(C₃H₆O)b(C₂H₄O)aH
Such copolymers are available from BASF Corporation under the Pluronic registered trade name.

The amount of phospholipids, by weight, in the emulsions of the present invention may be within a range of about 1% to about 5% by weight (including any value within this range).

The compositions of the present invention may also contain minor additives such as compounds to adjust tonicity, such as glycerol (about 1% to about 5% (w/w)), mannitol (about 1% to about 5% (w/w)), sucrose (about 2% to about 10% (w/w)), glucose (about 1% to about 10% (w/w)), glycine (about 1% to about 10% (w/w)) and/or propylene glycol (about 1% to about 10% (w/w)).

The compositions of the present invention may optionally contain stabilizing agents (referred to as “stabilizers”) to prevent or reduce the deterioration of the other components in oil-in-water emulsions, including antioxidants (e.g., glycine, α-tocopherol or ascorbate), or to prevent or inhibit microbial growth in the emulsions (e.g., EDTA) (including any value within this range). In certain embodiments, the concentration of glycine is about 0.1% to about 5% (e.g., about 1%) by weight. In certain embodiments, the concentration of EDTA is about 0.001% to about 0.01% (including any value within this range) by weight.

The aqueous phase of an oil-in-water emulsion of the present invention is usually at a concentration of at least about 70% by weight of the emulsion composition. In certain embodiments, the aqueous phase is at a concentration of at least about 75%, 80% or 85%, by weight of the emulsion composition.

The pH of the aqueous phase of the present compositions is generally between about 5 and 8 (including any value within this range).

In certain embodiments, the oil-in-water emulsions of the present invention are stable both chemically and physically. An oil-in-water emulsion is “physically stable” if it may be stored under appropriate conditions (e.g., at 2-8° C. or 25° C.) for at least 1 month without increase in average droplet size by more than 100%, or evidence of phase separation or oil droplet aggregation (coalescence). In certain embodiments, the average size of oil droplets of an emulsion of the present invention does not increase by more than about 10%, 20%, 25%, 30%, 40%, 50%, 75%, 100%, 125%, 150%, 175%, or 200% under appropriate storage conditions (e.g., at 2-8° C. or 25° C.) for at least 1, 2, 3, 4, 5, 6, 9, 12, 15, 18, or 24 months.

An oil-in-water emulsion is “chemically stable” if the elemene concentration in the emulsion does not change by about 20% under appropriate storage conditions for at least 1 month. In certain embodiments, the elemene concentration in an emulsion of the present invention does not change by about 5%, 10%, 15% or 20% under appropriate storage conditions for at least 1, 2, 3, 4, 5, 6, 9, 12, 15, 18, or 24 months.

In certain embodiments, the oil droplets of the oil-in-water emulsions are of sub-micron size. A “sub-micron size droplet” refers to an oil droplet in an oil-in-water emulsion having an average diameter of less than 1 micron as measured by conventional sizing techniques such as laser light scattering spectrometry. In certain embodiments, the oil droplets of the compositions of the present invention have an average diameter of less than about 500, 450, 400, 350, 300, or 250 nm. Oil droplets of sub-micron size are desired for the safe passage of these droplets in the capillary blood vessel in the circulation. Droplets of greater than 5 micron in diameter are believed to be unsafe for intravenous injection since they may block the capillary blood vessel resulting in pulmonary embolism. In certain embodiments, the oil droplets of the compositions of the present invention have an average diameter of less than 0.2-micron (200 nm) so that the emulsion may be sterilized by filtering through a 0.2 micron sized filter membrane. In certain embodiments, the oil droplets of the compositions of the present invention have an average diameter of less than about 150, 100, 75, 50, 25, 20, 15, or 10 nm.

In certain embodiments, the oil-in-water emulsions of the present invention have a wide range of temperature stability (e.g., −20° C. to 40° C.). In certain embodiments, the oil-in-water emulsions are stored at about 5° C. to about 25° C., or about 2° C. to about 8° C.

In certain embodiments, some or all of the components other than the elemene in the oil-in-water emulsion (e.g., an oil component, an emulsifier, a stabilizer, and a tonicity modifier) is safe, well tolerated, and acceptable by the FDA for intravenous injection.

A component of oil-in-water emulsions is regarded as “safe” if it does not cause significant undesired systemic reactions such as anaphylactic shock in patients.

A component of oil-in-water emulsions is regarded as “well tolerated” if it does not result in substantially adverse effects at the injection site, such as phlebitis, vein inflammation or vein irritation.

A component of an oil-in-water emulsion is regarded as “acceptable by the FDA” if it has been used in intravenous injection products approved by the FDA as of the filing date of the present application, and is being used at a concentration comparable to those used in FDA approved products.

In certain embodiments, some or all of the components other than the elemene in the oil-in-water emulsion (e.g., an oil component, an emulsifier, a stabilizer, and a tonicity modifier) is generally regarded as safe for use in intravenous injections by a drug regulatory authority.

A component of oil-in-water emulsion is “generally regarded as safe for use in intravenous injections by a drug regulatory authority” if it has been used in intravenous injection products approved by the FDA or a drug regulatory authority in Europe as of the filing date of the present application, and is being used at a concentration comparable to those used in the products approved by the FDA in the United States or by a drug regulatory authority in Europe.

In certain embodiments, the oil-in-water emulsions of the present invention are vein non-irritable. “Vein non-irritable” refers to the property of a compound or composition, when administered intravenously, does not cause substantial irritation at the injection site, as evident by, for example, thickened skin, necrotic skin, local redness, local swelling, venous dilation with blood clog formation, or venous embolism with subcutaneous inflammation.

In certain embodiments, the present invention provides an injectable oil-in-water emulsion comprising about 0.5 mg/mL to 50 mg/mL elemene(s), about 1% to about 20% (w/w) triglyceride oil(s), about 1% about 5% emulsifier(s), and about 1% to 10% (w/w) tonicity-adjusting agent(s), and water.

In certain embodiments, the present invention provides an injectable oil-in-water emulsion comprising about 0.1% to about 5% (w/w) elemene(s), about 1% to 10% (w/w) triglyceride oil(s), about 1% to about 5% emulsifier(s), and about 1% to 5% (w/w) tonicity-adjusting agent(s), and water.

In certain embodiments, the present invention provides an injectable oil-in-water emulsion comprising about 0.5% (w/w) elemene(s), about 5% (w/w) long chain triglyceride oil(s), about 5% (w/w) medium chain triglyceride oil(s), and about 1% to about 3% phospholipid(s), and about 1% to 3% (w/w) tonicity-adjusting agent(s), and water.

In certain embodiments, the present invention provides an injectable oil-in-water emulsion comprising about 0.5% (w/w) elemene(s), about 5% (w/w) vegetable oil(s), about 5% (w/w) medium chain triglyceride oil(s) (e.g., Miglyol 812), and about 1% to about 3% lecithin (e.g., egg lecithin or soy lecithin), and about 1% to 3% (w/w) tonicity-adjusting agent(s) (e.g., glycine or glycerol), and water.

In certain embodiments, the present invention provides an injectable oil-in-water emulsion comprising about 0.5% by weight elemene(s), about 10% by weight soybean oil, about 3% by weight egg or soy phospholipid, about 2.2% by weight glycerol, and water. The pH of the aqueous phase of the emulsions is from about 5 to about 8.

In certain embodiments, the present invention provides an injectable oil-in-water emulsion comprising about 0.5% by weight elemene(s), about 10% by weight soybean oil, about 1.2% to about 1.8% by weight egg or soy phospholipid, about 2.2% by weight glycerol, and water. The pH of the aqueous phase of the emulsions is from about 5 to about 8.

In certain embodiments, the present invention provides an injectable oil-in-water emulsion comprising about 0.5% by weight elemene(s), about 5% by weight soybean oil, about 5% medium chain triglyceride, about 3% by weight egg or soy phospholipid, about 2% by weight glycerol, about 1% glycine and water. The pH of the aqueous phase of the emulsions is from about 5 to about 8.

In certain embodiments, the present invention provides an injectable oil-in-water emulsion comprising about 0.5% by weight elemene(s), about 10% by weight soybean oil, about 1.2% to about 1.8% by weight egg or soy phospholipid, about 2.2% by weight glycerol, about 0.24% oleic acid, and water. The pH of the aqueous phase of the emulsions is from about 5 to about 8.

The present compositions may be prepared by appropriate methods known in the art, as well as those described in the examples below. An exemplary method for preparing the elemene oil-in-water emulsions according to the present invention may include the following general steps:
1. Mix appropriate amount of an emulsifier and a liquid oil;
2. Dissolve elemene(s) in the mixture resulting from step 1;
3. Dissolve water soluble components (e.g., glycine and glycerol) of the emulsion in water;
4. Mix the mixtures resulting from steps 2 and 3 to form an oil-in-water emulsion; and
5. Filter sterilize the oil-in-water emulsion resulting from step 4.

In certain embodiments, steps 1 and 2 may be combined together. In other words, both elemene and the emulsifier may be added to and dissolved in the liquid oil at the same time.

Besides being ready-to-use oil-in-water emulsions, the elemene compositions of the present invention can also be prepared with a cryoprotectant(s) as a lyophilized solid, i.e., “an oil-in-solid dispersion system” that can be reconstituted at a later date and diluted with water to reform the oil-in-water emulsion before injection.

As used herein, the term “an oil-in-solid dispersion system” refers to a solid matrix prepared by freeze-drying (lyophilizing) an oil-in-water emulsion of the present invention, which can reform an oil-in-water emulsion of similar droplet size upon mixing with water (reconstitution). In certain embodiments, the average droplet size of the reformed emulsion is no more than about 500%, 400%, 300%, 200%, or 150% of the average droplet size of the emulsion before the freeze-drying. An oil-in-solid dispersion system of this invention may be optionally prepared by spray drying.

“Cryoprotectants” used in the emulsion compositions of the present invention refers to those ingredients which are added to maintain the discrete and submicron droplets of the emulsion during the freeze-drying process and, upon the removal of water of the emulsion, to provide a solid matrix for the droplets to form the an oil-in-solid dispersion system.

Cryoprotectants that may be used in the emulsion compositions of this invention include, but are not limited to, polyols, monosaccharides, disaccharides, polysaccharides, amino acids, peptides, proteins, and hydrophilic polymers, or mixtures thereof.

Polyols that may be used in the present invention include, but are not limited to, glycerin, mannitol, erythritol, maltitol, xylitol, sorbitol, polyglycitol or mixtures thereof.

Monosaccharides that may be used in this invention include, but are not limited to, glucose, mannose, fructose, lactulose, allose, altrose, gulose, idose, galactose, talose, ribose, arabinose, xylose, lyxose or mixtures thereof.

Disaccharides that may be used in this invention include, but are not limited to, sucrose, lactose, maltose, isomaltose, trehalose, cellubiose or mixtures thereof.

Polysaccharides that may be used in this invention include, but are not limited to, cellulose, amylose, inulin, chitin, chitosan, amylopectin, glycogen, pectin, hyaruronic acid or mixtures thereof.

Amino acids that may be used in this invention include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine or mixtures thereof.

Peptides that may be used in this invention include, but are not limited to, diglycine and triglycine.

Proteins that may be used in this invention include, but are not limited to, albumin, collagen, casein, and gelatin.

Hydrophilic polymers that may be used in this invention include, but are not limited to, polyethylene glycols povidones, poloxamers, polyvinyl alcohols or mixtures thereof. The most preferred hydrophilic polymers are polyethylene glycols and povidones.

The concentration of the cryoprotectants used in the liquid emulsion compositions may be in the range of about 2% to about 40% w/w, such as about 5% to about 20% w/w and about 10% to about 15% w/w.

The present compositions may be used in ameliorating diseases, disorders or conditions that are susceptible to elemene treatment. Such diseases, disorders or conditions include, but are not limited to, burn, mosquito bites, Herpes Simplex infection, malignant pleural and peritoneal effusions, cancer (e.g., brain tumors, cancer of respiratory and digestive tracks, cancer of female reproductive organs, breast cancer, metastatic bone cancer, skin cancer, lymphoma, and leukemia). The compositions can be delivered by any parenteral route, including but not limited to, intramuscular, intravenous, intradermal, and peritoneal routes. In certain embodiments, the compositions may be directly injected into a solid tumor.

In one aspect, the present invention provides a method for reducing neoplasm in mammals (e.g., human) by administering to a mammal in need thereof a therapeutically effective amount of elemene in the compositions described herein. Reducing neoplasm is understood to encompass reducing or eliminating the occurrence, growth or progression of neoplasm. A mammal in need thereof may be a human and non-human mammal that is at risk for developing neoplasm or already has neoplasm.

The effectiveness of a given composition according to the present invention may be evaluated using in vitro cultured tumor cells or other cells (e.g., virus-transfected cells). Alternatively, the effectiveness may be determined using in vivo animal models and/or patients.

The following examples are intended to illustrate the invention without limiting the practice thereof.

### EXAMPLES

### Example 1

A representative composition of the present invention was prepared as follows:

TABLE 1
An Exemplary Composition and Components Thereof
(for a 1000 g batch)

| | | | Emulsion | Emulsion | | |
|---|---|---|---|---|---|---|
| | | | containing | containing No | | |
| | | | Elemenes | Elemenes | Active | Placebo |
| | | | (Active) | (Placebo) | g/1000 g | g/1000 g |
| Component | Grade | Manufacturer | % w/w | % w/w | batch | batch |
| Elemenes | Contains >70% | Dalian | 0.5 | 0 | 5 | 0 |
| | beta- | JinGong | | | | |
| | elemene | Pharma, | | | | |
| | | China | | | | |
| Egg lecithin | EP | LIPOID | 3 | 3 | 30 | 30 |
| (Lipoid E-80) | | | | | | |
| Medium | EP | SASOL | 5 | 5 | 50 | 50 |
| chain | | | | | | |
| triglyceride | | | | | | |
| oil (Miglyol | | | | | | |
| 812) | | | | | | |
| Soybean oil, | USP | Croda | 5 | 5 | 50 | 50 |
| super- | | | | | | |
| refined | | | | | | |
| Glycine free | USP | JT Baker | 1 | 1 | 10 | 10 |
| base | | | | | | |
| Glycerol | USP/NF | Dow | 2 | 2 | 20 | 20 |
| | | Chemicals | | | | |
| Water | De-ionized | | Add up to | Add up to | Add up to | Add up to |
| | water | | final | final | final | final |
| | | | volume | volume | volume | volume |
| Total | | | 100 | 100 | 1000 | 1000 |

Procedures for Preparing the Exemplary Composition

(a) Weigh out Lipoid E-80, Miglyol 812 and soybean oil into a clean 1000 mL round bottom flask.

(b) Rotate the flask using a rotovap to dissolve solids to form an oil phase.

(c) Add elemenes, rotate to dissolve elemenes in the oil phase.

(d) Sparge deionized water with filtered nitrogen gas (NF) for 10 minutes. Add the nitrogen sparged water to the flask.

(e) Add glycine and glycerol into the flask.

(f) Dissolve all solids by rotation to form an aqueous phase.

(g) Using a high-shear mixer (Ultra-Turrax Model 1810), mix the elemene-containing oil phase and the aqueous phase for 5 minutes to obtain a primary emulsion.

(h) Adjust pH to between pH 6 and 7 using HCl or NaOH.

(i) Filter the primary emulsion through a 5-micron filter.

(j) Pass the primary emulsion through a Microfluidizer (Model 110F equipped with an emulsion interaction chamber and operated at 18,000 PSI) for 5 passages to obtain a final emulsion.

(k) Aseptically, filter through a 5-micron filter and then through a sterile 0.2-micron filter.

(j) Determine the average droplet diameter by laser light scattering technique, the presence of large droplets (>5 micron or 5000 nm) by optical microscope, and the elemene concentration by an HPLC method.

The average droplet diameter was found to be 163 nm, the concentration of elemene was 5.3 mg/mL, and no large droplets (>5 micron or 5000 nm) were present in the emulsion.

### Example 2

TABLE 2
An Exemplary Composition and Component Thereof
(for a 1000 g batch)

| | | | Emulsion | Emulsion | | |
|---|---|---|---|---|---|---|
| | | | containing | containing No | | |
| | | | Elemenes | Elemenes | Active | Placebo |
| | | | (Active) | (Placebo) | g/1000 g | g/1000 g |
| Component | Grade | Manufacturer | % w/w | % w/w | batch | batch |
| Elemene | Contains >70% | Dalian | 0.5 | 0 | 5 | 0 |
| | beta | JinGong | | | | |
| | elemene | Pharma, | | | | |
| | | China | | | | |
| Soy lecithin | EP | Phospholipid | 1.2 | 1.2 | 12 | 12 |
| (Phospholipon | | Gmbh | | | | |
| 90G) | | | | | | |
| Medium chain | EP | SASOL | 5 | 5 | 50 | 50 |
| triglyceride oil | | | | | | |
| (Miglyol 812) | | | | | | |
| Soybean oil, | USP | Croda | 5 | 5 | 50 | 50 |
| super-refined | | | | | | |
| Glycine free | USP | JT Baker | 1 | 1 | 10 | 10 |
| base | | | | | | |
| Glycerol | USP/NF | Dow | 2 | 2 | 20 | 20 |
| | | Chemicals | | | | |
| Water | De-ionized | | Add up to | Add up to | Add up to | Add up to |
| | water | | final | final volume | final | final |
| | | | volume | | volume | volume |
| Total | | | 100 | 100 | 1000 | 1000 |

The compositions of EXAMPLE 2 were prepared using the same procedure as in EXAMPLE 1. The average droplet diameter was 467 nm.

### Example 3

TABLE 3
An Exemplary Composition and Components Thereof
(for a 1000 g batch)

| | | | Emulsion | Emulsion | | |
|---|---|---|---|---|---|---|
| | | | containing | containing No | | |
| | | | Elemene | Elemene | Active | Placebo |
| | | | (Active) | (Placebo) | g/1000 g | g/1000 g |
| Component | Grade | Manufacturer | % w/w | % w/w | batch | batch |
| Elemene | Contains >70% | Dalian | 0.5 | 0 | 5 | 0 |
| | beta | JinGong | | | | |
| | elemene | Pharma, | | | | |
| | | China | | | | |
| Egg lecithin | EP | LIPOID | 1.2 | 1.2 | 12 | 12 |
| (Lipoid E-80) | | | | | | |
| Soybean oil, | USP | Croda | 10 | 10 | 100 | 100 |
| super- | | | | | | |
| refined | | | | | | |
| Glycine free | USP | JT Baker | 1 | 1 | 10 | 10 |
| base | | | | | | |
| Glycerol | USP/NF | Dow | 2 | 2 | 20 | 20 |
| | | Chemicals | | | | |
| Water | De-ionized | | Add up to | Add up to | Add up to | Add up to |
| | water | | final | final | final | final |
| | | | volume | volume | volume | volume |
| Total | | | 100 | 100 | 1000 | 1000 |

The compositions of EXAMPLE 3 can generally be prepared using the same procedure as in EXAMPLE 1.

### Example 4

Stability Test

The compositions prepared according to EXAMPLE 2 were tested for stability. The stability parameters included elemene concentrations measured by a HPLC method, average droplet diameters measured by laser light scattering techniques, counts of large droplets (>5-micron or 5000 nm) and pH.

| | Elemene Concentration | | |
|---|---|---|---|
| Storage | (% w/w) | | |
| Condition | Initial | 3 month | 6.5 month |
| −20° C. | 5.88 | 5.596 | 4.764 |
| 5° C. | | 5.648 | 4.713 |
| 25° C. | | 5.016 | 4.334 |
| 40° C. | | 3.490 | 2.368 |

| | Average droplet | | |
|---|---|---|---|
| Storage | diameter (nm) | | |
| condition | Initial | 3 month | 6.5 month |
| −20° C. | 129 | 200 | >1000 |
| 5° C. | | 131 | 120 |
| 25° C. | | 136 | 130 |
| 40° C. | | 185 | 204 |

| | Counts of large | | |
|---|---|---|---|
| Storage | droplets (>5 micron) | | |
| condition | Initial | 3 month | 6.5 month |
| −20° C. | None | Many | Many |
| 5° C. | | None | None |
| 25° C. | | None | None |
| 40° C. | | None | Some |

The following compositions were also prepared and tested for stability as described above.

| Component | % w/w |
|---|---|
| Elemene | 0.5 |
| Egg lecithin (Lipoid E80) | 3 |
| Miglyol 812 | 5 |
| Soybean Oil, super refined | 5 |
| Glycine free base | 1 |
| Glycerol | 2 |
| EDTA, Disodium | 0.005 |
| Water | 83.5 |
| Total | 100 |

Elemene Concentrations Measured by HPLC

| | |
|---|---|
| Storage | Concentration (mg/mL) |

| Temperature | Initial | 4 month | 6 month | 12 month |
|---|---|---|---|---|
| −20° C. | 5.302 | 5.568 | 5.083 | 5.323 |
| 2-8° C. | | 5.557 | 5.153 | 5.574 |
| 25° C. | | 5.626 | 5.161 | 5.183 |
| 40° C. | | 5.477 | 4.775 | 0.753 |

Average Droplet Diameters Measured by Laser Light Scattering Technique

| | |
|---|---|
| Storage | Average droplet diameter (nm) |

| Temperature | 0 | 4 month | 6 month | 12 month |
|---|---|---|---|---|
| −20° C. | 129 | 200 | 340 | 416 |
| 2-8° C. | | 131 | 135 | 172 |
| 25° C. | | 136 | 125 | 174 |
| 40° C. | | 185 | 114 | 259 |

pH of the Emulsion Measured by a pH Meter

| | |
|---|---|
| | Average droplet |
| Storage | diameter (nm) |

| Temperature | 0 | 3 Month |
|---|---|---|
| −20° C. | 7.5 | 7.5 |
| 2-8° C. | | 7.5 |
| 25° C. | | 7.1 |
| 40° C. | | 6.9 |

Conclusion: The compositions prepared according to this invention appeared stable at 2-8° C. or 25° C. or in the refrigerator.

### Example 5

### Determining the Degree of Elemene Incorporation in the Emulsion Oil Phase

The degree of elemene incorporation in the emulsion droplets was measured by separating oil phase from aqueous phase of the emulsion and then measuring the concentration of elemene in the aqueous phase by HPLC. The separation of the oil phase and aqueous phase was accomplished by filtering the emulsion through a centrifugal filter with 10K molecular weight cutoff pore size (Microcon YM-10K Prod#42407 by Millipore Corp., MA). This filter allows the aqueous phase to pass through, but retains the oil phase. The elemene concentration found in the aqueous phase represents the un-incorporated (“unshielded”) elemene. A low concentration of elemene in the aqueous phase is desired since the un-incorporated (“unshielded”) elemene may be the cause of vain irritation. The compositions tested in this study included an elemene emulsion prepared according to this invention (Example 2) and the marketed elemene emulsion (“Elemene Emulsion for Injection”) by Dalian JinGong Pharmaceutical Co., Ltd., China.

TABLE 8
Degree of elemene incorporation

| | Elemene |
|---|---|
| | concentration in the |
| | aqueous phase |
| Compositions tested | (μg/mL) |
| An Elemene emulsion prepared | 7 |
| according to this invention (Example 2) | |
| “Elemene Emulsion for Injection” by | 39 |
| Dalian JinGong Pharmaceutical Co., | |
| Ltd., China. | |

The marketed elemene formulation (“Elemene Emulsion for Injection”) by Dalian JinGong Pharmaceutical Co., Ltd., China appeared to have a much higher concentration of free elemene in the aqueous phase.

### Example 6

### Vein Irritation Test

Elemene in the emulsion prepared according to Example 1 (5.3 mg/mL) was evaluated for vein irritation in rabbits. The marketed product (“Elemene Emulsion for Injection” by Dalian JinGong Pharmaceutical Co., Ltd., China) was also tested for comparison. Normal saline was used as a negative control.

Methods: Nine (9) New Zealand white rabbits were randomly divided into 3 groups and infused at a constant rate through marginal ear vein with the elemene emulsion prepared according to Example 1 (“EE”), the marketed Elemene product (MEP), or normal saline followed by appearance and pathology examinations for venous irritation reactions near the injection site. EE group (n=3): EE at 5.3 mg/ml was infused at 1.0 mL/min and 30 mL/animal per day for 3 days; MEP group (n=3): MEP at 5.0 mg/mL was infused at 1.0 mL/min and 30 mL/animal per day for 3 days; Control group (n=3): 0.9% sodium chloride for injection was infused at 1.0 mL/min and 30 mL/animal per day for 3 days. Pathology examination was conducted at 48 h after the last injection.

Results: Appearance observation: In the MEP group, 24 hour after the first injection, severe ear vein irritation was observed with thicken and necrotic skin accompanied local redness and swelling in all 3 rabbits. No difference in rabbit marginal ear veins was observed between the EE group and the normal saline control group.

Pathology examination (marginal ear vein slice with HE stain): subcutaneous hemorrhage and inflammation were observed in the MEP group (3 rabbits). Venous embolism was seen in 1 of 3 MEP rabbits, venous endothelium necrosis accompanied local epidermal erosion and white blood cells embolism was seen in 1 of 3 MEP rabbits. No evidence of irritation to the venous endothelium was seen in EE group and the normal saline control group.

Conclusion: The marketed product of elemene (MEP) exhibited severe vein irritation, and the elemene emulsion prepared according to Example 1 exhibited the same vascular compatibility as the normal saline and is not vein irritating.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An injectable oil-in-water emulsion comprising a therapeutically effective concentration of elemene(s), at least one liquid oil, and water.

2. The emulsion according to claim 1 wherein the liquid oil is selected from the group consisting of a triglyceride, a diglyceride, a monoglyceride, a mixture of diglyceride and monoglyceride, vitamin E, and a derivative of vitamin E.

3. The emulsion according to claim 1 additionally comprising an emulsifier.

4. The emulsion according to claim 3 wherein the emulsifier is an egg or soy phospholipid.

5. The emulsion according to claim 2 wherein the triglyceride oil is an oil ester of C6 to C22 aliphatic fatty acids.

6. The emulsion according to claim 2 wherein the monoglyceride or diglyceride is an ester of C6 to C12 saturated fatty acid or C16 to C18 unsaturated fatty acid.

7. The emulsion according to claim 2 wherein the vitamin E or the derivative thereof is selected from alpha tocopherol and alpha tocopherol acetate.

8. The emulsion according to claim 1 wherein the elemene(s) contain greater than 50% beta-elemene based on the weight of all elemene isomers present, and wherein betal-elemene has the structure:

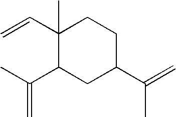

9. The emulsion according to claim 8 wherein the elemene(s) contain greater than 80% beta-elemene based on the weight of all elemene isomers present.

10. The emulsion according to claim 8 wherein the elemene(s) contain greater than 90% beta-elemene based on the weight of all elemene isomers present.

11. The emulsion according to claim 1 wherein the therapeutically effective concentration of elemene(s) is from about 0.5 mg/ml to about 50 mg/ml.

12. The emulsion according to claim 1 wherein the therapeutically effective concentration of elemene(s) is about 5 mg/ml.

13. The emulsion according to claim 1 additionally comprising a tonicity-adjusting agent.

14. The emulsion according to claim 13 wherein the tonicity-adjusting agent is selected from the group consisting of propylene glycol, glycerol, mannitol, sorbitol, xylitol, sucrose, glucose, fructose, trehalose, lactose, glycine, dextrin, sodium chloride and a mixture thereof.

15. The emulsion according to claim 1 additionally comprising disodium edetate.

16. The emulsion according to claim 1 wherein the liquid oil is a mixture of long chain triglyceride and medium chain triglyceride, and wherein the emulsion further comprises phospholipid.

17. The emulsion of claim 16 further comprising a tonicity-adjusting agent, disodium edetate, or both a tonicity-adjusting agent and disodium edetate.

18. An injectable oil-in-water emulsion comprising about 0.5% (w/w) elemene(s), about 5% (w/w) vegetable oil, about 5% (w/w) medium chain triglyceride, about 1% (w/w) to 3% (w/w) phospholipid, about 1% (w/w) glycine, about 2% (w/w) glycerol, and water.

19. A method of reducing neoplasm in mammals comprising administering to a mammal in need thereof a therapeutically effective amount of elemene(s) in the emulsion according to claim 1.

20. The method according to claim 19 wherein the neoplasm is selected from the group consisting of leukemias, sarcomas, carcinomas, and myelomas.
